(19) United States
(12) Patent Application Publication
(10) Pub. No.: US 2003/0157050 A1
(43) Pub. Date: Aug. 21, 2003

(54) Cosmetic lotions comprising cocoa butter

(76) Inventors: Helen Ambrosen, Dorset (GB); Mark Constantine, Dorset (GB); Margaret Constantine, Dorset (GB)

Correspondence Address: Judson K Champlin, Westman Champlin & Kelly, International Centre Suite 1600, 900 Second Avenue South, Minneapolis, MN, 55402-3319, US

(21) Appl. No.: 10/258,680

(22) PCT Filed: Apr. 27, 2001
(86) PCT No.: PCT/GB01/01870

(30) Foreign Application Priority Data
Apr. 28, 2000 (GB) 0010425.7

(51) Int. Cl. A61K 7/06; A61K 35/78

(52) U.S. Cl. 424/74; 424/776

(57) ABSTRACT

A cosmetic lotion comprising 16% to 76% by weight cocoa butter and having a solid rather than liquid form. Also a method of manufacturing a cosmetic lotion having an oil based component and a water based component characterised by selecting the oil based component to comprise cocoa butter so that the cocoa butter constitutes 16% to 76% by weight of the final form of the lotion; heating the cocoa butter to a temperature in the range 55° to 70° C.; cooling the cocoa butter to a temperature in the range 35° to 25° C. and at a temperature in that range adding the water based component.

The present invention relates to cosmetic lotions.

Cosmetic lotions are useful preparations for softening and moisturising the skin. They are commonly made from oil based ingredients and water based ingredients held together in suspension by an emulsifier. Without an emulsifier the oil and water phases would separate. Lotions are flowing, liquid preparations. They are normally packaged in a rigid container made of glass or plastic which is often not biodegradable.

According to a first aspect of the present invention there is provided a cosmetic lotion comprising 16% to 76% by weight cocoa butter and having a solid rather than liquid form.

According to a second aspect of the present invention there is provided a method of manufacturing a cosmetic lotion having an oil based component and a water based component characterised by selecting the oil based component to comprise cocoa butter so that the cocoa butter constitutes 16% to 76% by weight of the final form of the lotion; heating the cocoa butter to a temperature in the range 55° to 70° C.; cooling the cocoa butter to a temperature in the range 35° to 25° C. and at a temperature in that range adding the water based component.

Embodiments of the present invention will now be described, by way of example only.

The present invention utilises the emulsifying properties of cocoa butter to form a novel emulsion system. It contains water based and oil based ingredients. These would normally separate into two phases. Using Cocoa Butter as an emulsifier holds the ingredients in suspension. The nature of Cocoa Butter is a solid and this gives the structure of a bar.

Cocoa Butter comes from the bean of the Cocao tree (Theobroma cacao), more commonly known as cocoa beans. The beans are fermented, roasted and ground to produce an oily paste. The cocoa fat, more than 50% of the bean, is rendered to produce cocoa butter. The remaining fat free, powered residue of the bean is cocoa.

In cosmetic products the emollient qualities of cocoa butter are used to soften the skin. However, the present invention is based on it's emulsifying qualities. Oil and water based ingredients are held together in suspension in the form of a bar. When applied to the skin the effect is the same as that of a lotion. Water based materials will moisturise and oil based ingredients will soften. After application, the skin feels moisturised but not heavily oiled as it would do if an oil-based cocoa butter bar had been applied.

The nature of Cocoa Butter is a solid and this forms the structure of the bar with the emulsion in situ. Many shapes and applications can be achieved because of this solid form.

The Solid Lotion Bar can be given further applications. By the addition of sunscreens it is be capable of protecting the skin from the sun. The addition of harder waxes such as beeswax modifies the melting point. The addition of a material such as sodium bicarbonate provides a deodorising product. The addition of clays such as talc gives a product capable of absorbing excess moisture on the skin or solid ‘talc’. Varying ingredients can be added to give formulae suitable for different skin types. Fruits and vegetables are compatible with the system. Their water based ingredients give the bar different applications, eg. Banana softens the skin, Garlic prevents the formation of spots on the skin.

An example of a cosmetic lotion according to the present invention is as follows:

### EXAMPLE OF A LOTION IN SOLID BAR FORM

| | % |
|---|---|
| Oil Based Ingredients | |
| Cocoa Butter | 70.0 |
| Almond Oil | 2.0 |
| Water Based Ingredients | |
| Fresh Banana or Avocado | 21.5 |
| Plant extract eg. Water based extract of Aloe Vera | 5.5 |
| Fragrance | 1.0 |
| | 100.0 |

According to the present invention the cocoa butter should constitute between 16% to 76% (inclusive) by weight of the final product. Preferably the cocoa butter should constitute between 30% to 75% (inclusive) by weight of the final product, more preferably 50% to 75% and most preferably 60% to 75%. Generally, it has been found that the advantages of the invention can be obtained using the following percentage ranges (by weight) of various desirable ingredients, namely:

### RANGE OF PERCENTAGES FOR INGREDIENTS IN SOLID LOTION BAR

| | % | | % |
|---|---|---|---|
| Oil Based Ingredients | | | |
| Cocoa Butter | 16.0 | - | 76.0 |
| Oil e.g. Almond | 10.5 | - | 1.0 |
| Water Based Ingredients | | | |
| Glycerine, Water of Water Based Plant Extract | 13.0 | - | 2.0 |
| Honey, Fresh Fruit or Vegetable | 60.0 | - | 20.0 |
| Fragrance | 0.5 | | 1.0 |
| | 100.0 | | 100.0 |

The manufacturing process of the bar is also of consequence. The Cocoa Butter and oils are melted to 55°-70° C. then cooled to 60°-25° C. The water based ingredients and fragrance are preferably added between 35° and 25° C. The mixture is then cooled to 30°-20° C. and poured into moulds. These are chilled to 20°-10° C. and hardened.

Heating Cocoa Butter to approximately 60° C. prevents undesirable crystallisation on cooling. Emulsifying the water based ingredients at approximately 30° C. ensures the emulsion forms fully. Cooling to approximately 25° C. ensures the bars exit the moulds easily when formed. Chilling at 16° C. allows the bars to cool quickly without cracking. Any substantial variation from these temperatures, at least with respect to the initial heating and the emulsifying stage, can result in a faulty final product.

As the bars are solid forms then can be packaged in biodegradable materials such as paper or cardboard. The bars can be treated by dipping in a molten hard wax such as beeswax or Japan wax to keep the hands clean when applying the bar to the body.

1. A cosmetic lotion comprising 16% to 76% by weight cocoa butter and having a solid rather than liquid form.

2. A cosmetic lotion as claimed in claim 1, comprising 30% to 75% by weight cocoa butter.

3. A cosmetic lotion as claimed in claim 1, comprising 50% to 75% by weight cocoa butter.

4. A cosmetic lotion as claimed in claim 1, comprising 60% to 75% by weight cocoa butter.

5. A cosmetic lotion as claimed in any preceding claim, wherein the lotion contains an oil based ingredient in addition to the cocoa butter.

6. A cosmetic lotion as claimed in claim 5, wherein the said oil based ingredient is almond oil.

7. A cosmetic lotion as claimed in any preceding claim, wherein the lotion contains a water based ingredient comprising part of a fruit.

8. A cosmetic lotion as claimed in any preceding claim, wherein the lotion contains a water based ingredient comprising part of a vegetable.

9. A cosmetic lotion as claimed in claim 1, comprising

| | (by weight) | | |
|---|---|---|---|
| | % | | % |
| Cocoa Butter | 16.0 | - | 76.0 |
| Oil e.g. Almond | 10.5 | - | 1.0 |
| Glycerine, Water or Water Based Plant Extract | 13.0 | - | 2.0 |
| Honey, Fresh Fruit or Vegetable | 60.0 | - | 20.0 |
| Fragrance | 0.5 | | 1.0 |

10. A cosmetic lotion as claimed in claim 1, comprising

| | (by weight) % |
|---|---|
| Cocoa Butter | 70.0 |
| Almond Oil | 2.0 |
| Fresh Banana or Avocado | 21.5 |
| Plant extract eg. Water based extract of Aloe Vera | 5.5 |
| Fragrance | 1.0 |

11. A method of manufacturing a cosmetic lotion having an oil based component and a water based component characterised by selecting the oil based component to comprise cocoa butter so that the cocoa butter constitutes 16% to 76% by weight of the final form of the lotion; heating the cocoa butter to a temperature in the range 55° to 70° C.; cooling the cocoa butter to a temperature in the range 35° to 25° C. and at a temperature in that range adding the water based component.

12. A method as claimed in claim 11, further comprising the step of: at a temperature in the range 30° to 20° C. pouring the mixed oil and water based components in to one or more moulds.

13. A method as claimed in claim 12, further comprising the step subsequent to said step of pouring of chilling the lotion to a temperature in the range 20° to 10° C.

14. A method as claimed in claim 11, comprising the steps of: heating the Cocoa Butter to approximately 60° C., emulsifying the water based ingredient at approximately 30° C., cooling the lotion to approximately 25° C., pouring the lotion in to one or more moulds and chilling the lotion at 16° C.
